(19) United States
(12) Patent Application Publication
(10) Pub. No.: US 2007/0298454 A1
(43) Pub. Date: Dec. 27, 2007

(54) Analysis Of Cell Morphology And Motility

(75) Inventors: Richard Benjamin Green, Glasgow (GB); Eric Augustine Gillies, Glasgow (GB); Richard Mchugh Cannon, Glasgow (GB); Alan Anthony Pacey, Sheffield (GB)

Correspondence Address: NIXON & VANDERHYE, PC, 901 NORTH GLEBE ROAD, 11TH FLOOR, ARLINGTON, VA, 22203, US

(73) Assignee: THE UNIVERSITY COURT OF THE UNIVERSITY OF GLASGOW, Glasgow, Strathclyde, GBN (GB)

(21) Appl. No.: 10/589,882

(22) PCT Filed: Feb. 16, 2005
(86) PCT No.: PCT/GB05/00558

§ 371 Date: Dec. 1, 2006

(30) Foreign Application Priority Data
Feb. 18, 2004 (GB) 0403611.7

(51) Int. Cl. C12Q 1/04; C12M 1/34

(52) U.S. Cl. 435/34; 435/287.1

(57) ABSTRACT

Determination of the morphology and motility of a population of cells in vitro is possible via a series of steps. The cells are imaged using a microscope and camera. A first frame of image data allows identification of parts of the image data corresponding to a cell or cells of interest. Cell morphology is determined from this data. A second frame of image data, captured subsequent to the first, allows the determination of the relative displacement of the cell or cells of interest. This provides motility data. The invention has particular application in male fertility investigations.

The present invention relates to a method and apparatus for analysing cell morphology and motility. It is particularly, but not necessarily exclusively, concerned with the analysis of the morphology and motility of spermatozoa, for example in male fertility investigations.

The analysis of human semen is currently a time consuming process that is prone to errors (Matson, 1995). Furthermore, it is difficult adequately to quality control such analysis (Clements et al., 1997). The technique of semen analysis is agreed by an international advisory board and the agreed technique is published by the World Health Organisation as a laboratory manual. The current version of the relevant WHO laboratory manual was published in 1999 and outlines the various macroscopic and microscopic measurements that should be made during human semen analysis (WHO, 1999). Whilst the macroscopic measures (for example seminal volume, pH, viscosity) are relatively straightforward, the three main microscopic measures (sperm concentration, sperm morphology and sperm motility) are performed in separate stages as explained below.

Sperm concentration is normally estimated using a haemocytometer. Other counting chambers may be used, although these are often considered inaccurate by comparison. To perform haemocytometry, sperm must be killed with fixative prior to counting.

Sperm motility measurements are made on live sperm observed on a microscope slide (or specialist observation chamber) of at least 20 μm depth. Up to 200 sperm are classified into one of four motility grades to determine the proportion of motile sperm in the sample.

Sperm morphology measurements (i.e. how many sperm are of the correct size and shape) are made on fixed, stained, killed sperm by smearing a small aliquot of semen onto a microscope slide and allowing it to dry before being fixed (using, e.g., methanol) and staining it with a histological stain (e.g. Papanicolau). Note that the sperm are killed through the fixing and staining process. At least 100 of the sperm are then observed and classified as normal or abnormal, or an index of abnormality is calculated e.g. using the TZ1 index (WHO, 1999).

In the majority of laboratories, the microscopic measures are made manually, with estimates of concentration and motility being made within an hour of ejaculation. However, measurements of sperm morphology can only be made once the smear has been stained and this may take several hours and can even be performed many days later.

Manual measurement of motility is usually undertaken by a technician visually examining a live cell sample under a microscope, attempting to count the number of motile cells in the field of view. This technique is unreliable as it is highly subjective, leading to different estimates of motility between different laboratories. Furthermore, the morphological determination is then made at a different time on a fixed, killed and stained sample.

To assist in these microscopic measurements, several manufacturers have developed and introduced into the market Computer Aided Sperm Analysers (CASA). See, for example, reference Mortimer, 1994 for a review. These CASA machines can improve the accuracy of the microscopic measures, although three major problems with them have been identified. Firstly, they have difficulty in tracking sperm trajectories which cross or where two or more sperm collide. This leads to broken tracks, which in turn gives rise to inaccurate kinematic statistics and elevated measurements of sperm concentration. Secondly, they are only able to generate reliable motility measurements within a relatively narrow range of sperm concentrations, as at high concentrations immotile sperm are jostled leading to elevated estimates of sperm motility. Thirdly, separate software is required to undertake the analysis of fixed sperm samples to provide an estimate of sperm morphology. Moreover, most current software has difficulty identifying defects of the sperm tail.

As a consequence of these drawbacks, and in addition to their relatively high purchase costs, CASA machines are rarely used in routine laboratories but remain the preserve of research institutions or specialist fertility centres.

The present inventors have realised that existing CASA machines fail to address a fundamental problem of semen analysis. This is that, to interpret the information from the microscopic measures, the clinician or researcher must assume that the motile sperm are morphologically normal, whereas in reality this is not always the case. The inventors have realised that it would be advantageous to provide a technique that can provide a single figure to determine the ‘concentration of motile morphologically normal sperm’ in an ejaculate and/or the frequency distribution of head parameters and/or kinematic data.

GB-A-2130718 discloses an apparatus for measuring spermatozoal motility. However, this apparatus is only capable of calculating an average velocity for the cells in a sample. It does not allow for the tracking of individual cells.

GB-A-2305723 discloses a cytological specimen analysis system. This system is analysing the morphology of killed cells.

WO92/13308 discloses a morphological classification method for cells. A digital representation of the cells is obtained and filtered to identify malignant or premalignant cells. However, the method is not carried out on live cells, so there can be no measurement of motility.

U.S. Pat. No. 4,896,967 discloses an apparatus for determining the motility if cells. Magnified images of live cells are captured using a video camera mounted on a microscope. The images are recorded for the purposes of motility analysis. There is no disclosure of morphological characterisation of the cells analysed.

It is known from a variety of physiological studies that the ability of sperm to pass through cervical mucus is dependent both upon its motility (Aitken et al., 1985; Mortimer et al., 1986) and morphological (Katz et al., 1990) characteristics. In addition, it has been suggested that only sperm with ‘normal’ morphology are able to ‘bind’ to the isthmic endosalpinx prior to fertilisation (Ellington et al., 1997) and subsequently bind to the zona pellucida of the egg (Liu & Baker, 1992). Consequently, the inventors consider that the ability to a) measure the concentration of motile sperm with normal morphology in an ejaculate (or a prepared sperm sample) and b) to define the frequency distribution of kinematic parameters as a function of head dimensions, could be a major advance in our ability to define the functional population of sperm. This is increasingly important with growing concern about the possible effects on semen quality of environmental or occupational factors (Swan and Elkin, 1999).

The present invention aims to address one or more of the above problems, preferably reducing, ameliorating or eliminating one or more of the above problems.

In a first aspect, the invention provides a method for determining the morphology and motility of a population of cells in vitro including the steps:
capturing a first frame of image data of said population and identifying a part or parts of the image data corresponding to a cell or cells of interest;
capturing a second frame of image data of said population and identifying a part or parts of the image data corresponding to a cell or cells of interest;
determining the morphology of the cell or cells of interest from the first and/or second frame; and
determining the relative displacement, in the second frame compared to the first frame, of the cell or cells of interest.

In this way, the inventors have found that it is possible to determine both the morphology and the motility of a population of cells. Known schemes, particularly those for analysis of semen samples, perform each test separately (the motility test being on a live sample, and the morphology test on a killed sample). An advantage that may be provided by the present invention is that the number of motile, morphologically normal cells, the frequency distribution of the head parameters and the kinematic properties can be determined in a single test.

Preferred and/or optional features will now be set out. These are applicable independently or in any combination with any aspect of the invention, unless the context demands otherwise.

Preferably, the first and second frames are adjacent frames in a series of more than two frames of image data captured of said population, the method further including, for each frame of said series, the steps:
identifying a part or parts of said image data corresponding to the cell or cells of interest; and
determining the relative displacement, in said frame compared to the previous frame in said series, of the cell or cells of interest.

Preferably, the method further including the step of determining the morphology of said cell or cells identified for each frame of said series.

Preferably, the method allows simultaneous determination of morphology and motility.

Preferably, the method includes the step of determining kinematic parameters for the motility of the cell or cells of interest, based on the relative displacement of the cell or cells of interest. Typically, the amount or relative amount of the population of cells having a motility at or above a threshold motility value may be determined.

Preferably, the method includes the step of classifying the cell or cells identified as morphologically normal or morphologically abnormal or making specific measurements of head size. Furthermore, the method may include the step of determining the amount or relative amount of the population of cells being morphologically normal.

In particular, the method may include the step of determining the amount or relative amount of the population of cells being morphologically normal and having a motility at or above a threshold motility value or as a frequency distribution.

Preferably, the method is for carrying out a first determination of morphology and motility on a first area of a sample of cells, the method including the step of carrying out a second and, optionally, further, determinations of morphology and motility on a second and, optionally, further, areas of the sample. In this way, the results from the method can be considered to be more representative than a test carried out on a single area alone.

In preferred embodiments, the method is carried out with the aid of image processing devices, usually using a suitably programmed computer. Typically, the human eye will be not be able to make determinations of morphology and motility with sufficient speed to provide reliable results.

The inventors realise that it may be possible to capture a series of frames of image data from the population of cells and, in a separate process, determine the morphology and motility of those cells using the image data. This constitutes an independent aspect of the invention.

Accordingly, in a second aspect, the invention provides a method of processing image data captured from a population of cells in vitro in order to determine the morphology and motility of the cells, the image data including

a first frame of image data of said population and a second frame of image data of said population, the method including the steps:
determining the morphology of the cell or cells of interest from the first and/or second frame; and
determining the relative displacement, in the second frame compared to the first frame, of the cell or cells of interest.

In the case where the morphological and motility determination is carried out on the image data separately from the capture of the image data, the image data may be stored in the intervening time on memory means, for example on the internal memory of a computer (ROM or RAM) or on an external memory means such as a portable data carrier (e.g. CD or DVD).

Preferred and/or optional features will now be set out. These are applicable independently or in any combination with any aspect of the invention, unless the context demands otherwise. In particular, it is intended that these features are also applicable to the first aspect.

Preferably, the first frame of image data is processed to identify illumination intensity distributions of interest having one of a plurality of characteristic profiles.

As a first example, one of the characteristic profiles may be a first characteristic profile having a centre point of a relatively high intensity surrounded by a substantially symmetrical gradual reduction in intensity. It has been found that such a profile is consistent with the illumination profile of the nucleus portion of the head of spermatozoa.

As a second example, one of the characteristic profiles is a second characteristic profile consistent with the illumination profile of the acrosome portion of the head of spermatozoa.

As a third example, one of the characteristic profiles is a third characteristic profile consistent with the illumination profile of the acrosome portion of the head of spermatozoa.

Preferably, the parts of the image data corresponding to the illumination intensity distributions of interest are further processed to identify cell perimeter features surrounding one or more of said illumination intensity distributions of interest. By looking for cell perimeter features in this way, it is possible to identify cells of interest because they have at least one illumination profile corresponding to a feature of interest (e.g. DNA portion, acrosome portion and/or nose portion) and a cell perimeter portion surrounding said illumination profile(s) of interest.

For example, the parts of the image data corresponding to the illumination intensity distributions of interest may be further processed to identify cell perimeter features surrounding an illumination intensity distribution having a characteristic profile with a centre point of a relatively high intensity surrounded by a substantially symmetrical gradual reduction in intensity.

In the case where the perimeter of the cells of interest (e.g. spermatozoa) themselves have a distinctive shape, the method may further include the step of processing the parts of the image data corresponding to the illumination intensity distributions of interest to identify cell perimeters of a characteristic shape.

Preferably, once an object of interest is identified, the method further includes the step of determining one or more dimensions or relative dimensions of the object. Said dimensions or relative dimensions may be compared to one or more predetermined ranges of corresponding dimensions or relative dimensions. For example, a look-up table may be used, the look-up table containing the predetermined ranges of dimensions or relative dimensions corresponding to cells of interest.

Preferably, the method further includes the step of determining whether said object is a cell to be tracked or not and, if said object is a cell to be tracked, assigning a tracking identity to it; or, if said object is not a cell to be tracked, assigning a residual object identity to it. In this way, substantially all of the objects identified in the image processing of the image data can have an identity assigned so that the same objects can be correctly identified in subsequent frames of image data.

Preferably, the method further includes the step of determining a characteristic morphological value for a cell to be tracked.

Typically, the method is repeated in order to identify all cells to be tracked and all objects not to be tracked in a frame of image data. Furthermore, this method may be repeated for the second and/or subsequent frames.

Having determined a characteristic morphology for the cells to be tracked, we can now look at how the cells may be tracked. Preferably, the method further includes tracking the cells by identifying said cells and their locations in the second and/or subsequent frames of image data. Typically, the tracking is carried out by collating the relative displacements of the locations of the cells through the sequence of frames of image data.

In the case where the tracks of two cells of interest intersect, the cells and their tracks are preferably identified before and after the intersection by their characteristic morphologies.

In the case where a cell of interest is identified in one frame and is not identified in the next frame, the cell then being identified in a subsequent frame, the method preferably further includes calculating tracking data to connect the track of the cell through said frames.

Preferably, the method further includes the step of determining a motility characteristic for a tracked cell.

The method may in particular include the determination of an overall figure of merit for the sample indicative of the number or proportion of morphologically normal cells with normal motility.

Additionally or alternatively, the method may include the step of processing data relating to motility and/or morphology of the cells identified in the population to provide statistical distributions of motility and/or morphology.

Typically, the image capture is performed using digital imaging means. The digital imaging means preferably provides a frame resolution or an effective frame resolution of at least 0.5×10⁶ pixels. The frame resolution or effective frame resolution may be of at least 10⁶ pixels. Preferably, the frame resolution or effective frame resolution is at least 5×10⁶ pixels.

Preferably, the digital imaging means has a pixel size of 10 mm×10 mm (or equivalent area for different shapes) or lower. For example, the pixel size may be 8 mm×8 mm or lower. Typically, the digital imaging means is used in combination with a microscope objective lens of at least ×20 (preferably ×40) magnification).

Typically, the rate of image capture for such a series of frames is at least 10 Hz. It may be at least 20 Hz, more preferably at least 30 Hz. In the case of spermatozoa, the beat cross frequency is typically 10 Hz, so image capture rates at least 20 Hz are preferred.

Preferably, the cell or cells of interest are spermatozoa, such as human spermatozoa.

In a third aspect of the invention, there is provided a method of diagnosis including a method according to the first or second aspect and a step of diagnosis based on the determination of the morphology and motility of the population of cells in vitro (or ex vivo).

Preferably, the step of diagnosis is based on a value of the amount or relative amount of cells categorised as morphologically normal and having a motility at or above a threshold motility value.

In a fourth aspect, there is provided apparatus for determining the morphology and motility of a population of cells in vitro or ex vivo, the apparatus including:
imaging means for capturing first and second frames of image data of said population and identifying a part or parts of the image data corresponding to a cell or cells of interest
computation means for determining the morphology of the cell or cells of interest from the first and/or second frame and for determining the relative displacement, in the second frame compared to the first frame, of the cell or cells of interest.

Preferred and/or optional features set out above may be applied independently or in any combination with this aspect of the invention.

Preferably, the apparatus is for carrying out a method of any one of the first, second or third aspects.

Preferably, the imaging means includes phase contrast optics.

In a fifth aspect of the invention there is provided a computer system operatively configured to carry out the method of any one of the first, second or third aspects.

In a sixth aspect of the invention there is provided computer programming code for operatively configuring a computer system to carry out the method of any one of the first, second or third aspects.

In a seventh aspect of the invention there is provided a data carrier having recorded on it computer programming code according to the sixth aspect.

Preferred embodiments of the invention will now be described, by way of example, with reference to the accompanying drawings, in which:

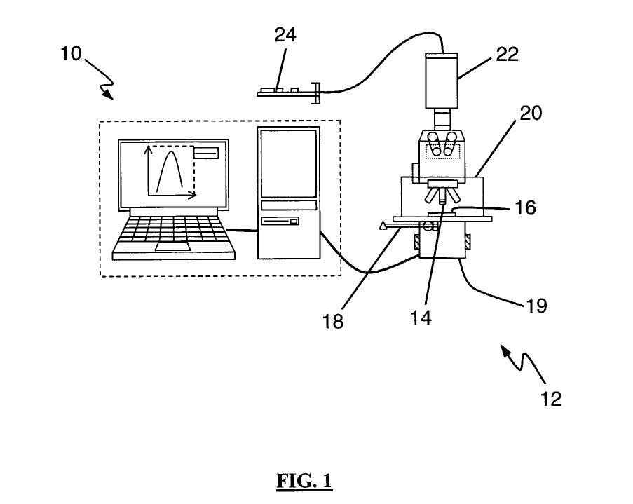

FIG. 1 shows a schematic view of an apparatus according to an embodiment of the invention.

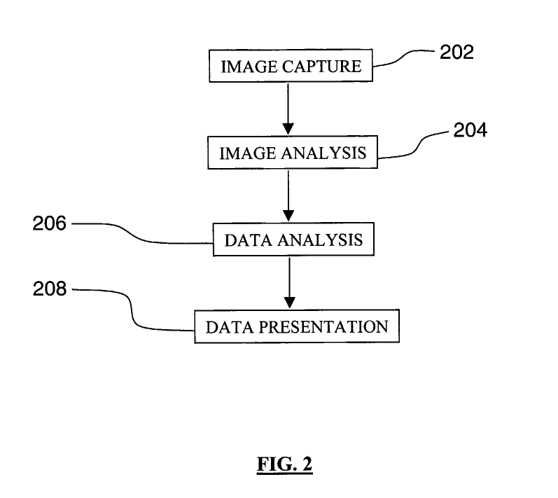

FIG. 2 shows a flow chart illustrating an overview of the functions performed by the apparatus of FIG. 1.

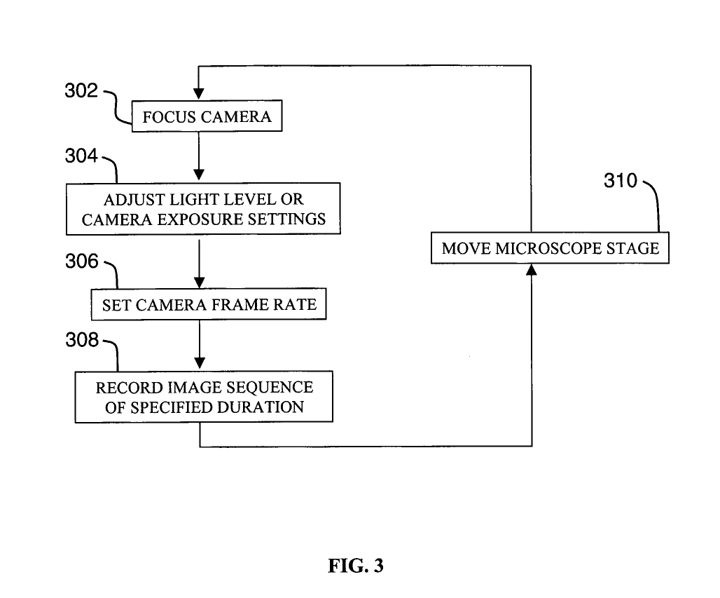

FIG. 3 shows a flow chart illustrating an image capture sequence for use in an embodiment of the invention.

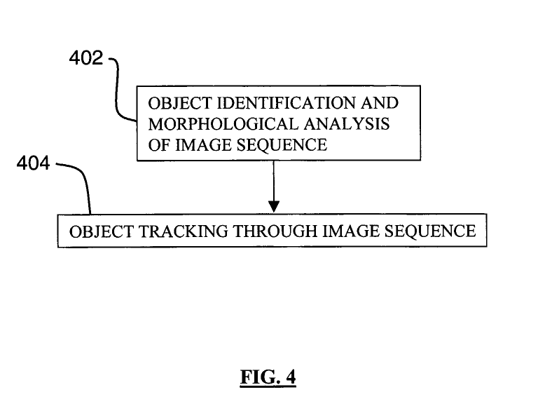

FIG. 4 shows a flow chart illustrating an overview of the image analysis methodology for use in an embodiment of the invention.

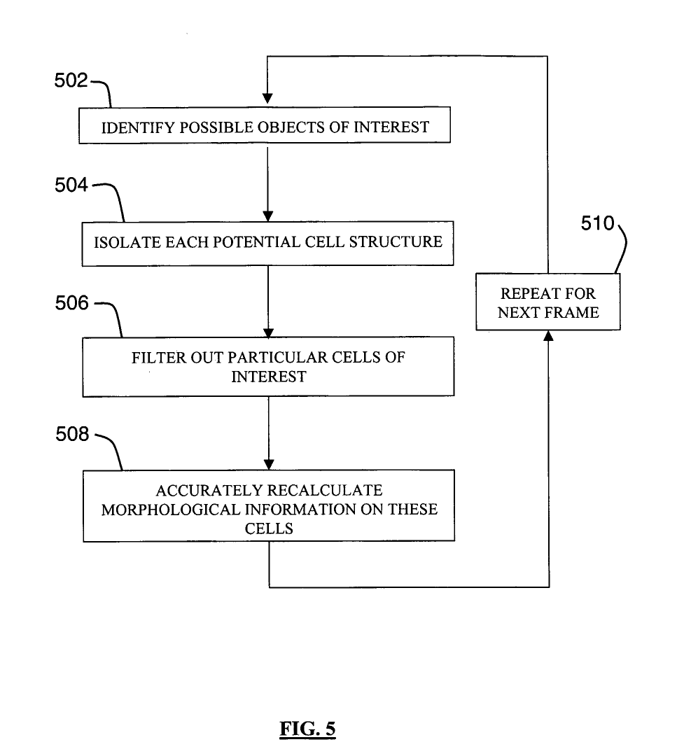

FIG. 5 shows a flow chart illustrating the morphological analysis methodology for FIG. 4.

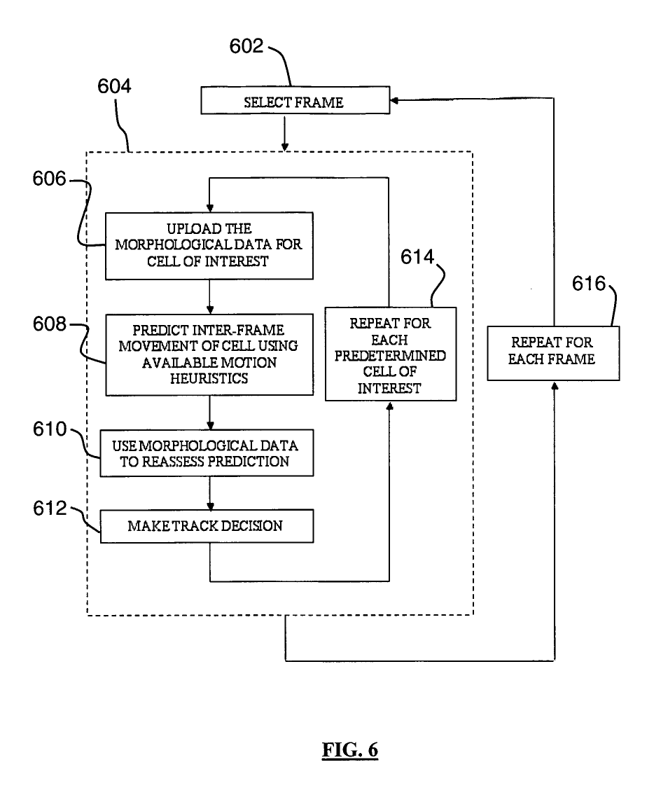

FIG. 6 shows a flow chart illustrating the tracking methodology for FIG. 4.

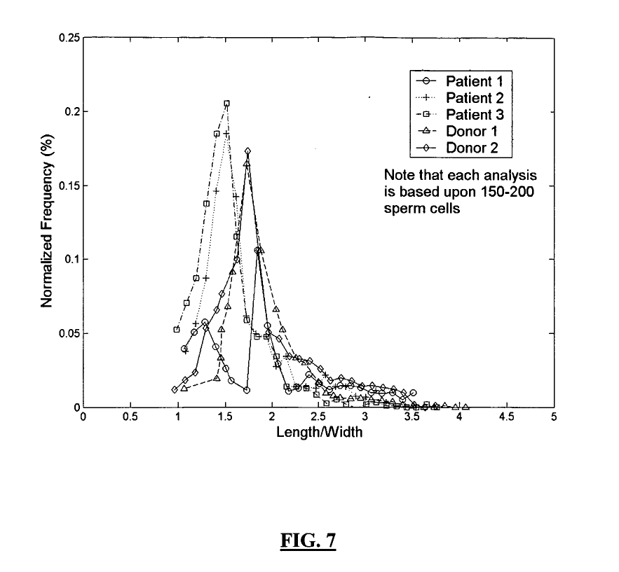

FIG. 7 shows a frequency distribution of the length/width ratio of motile sperm using an embodiment of the present invention on live samples.

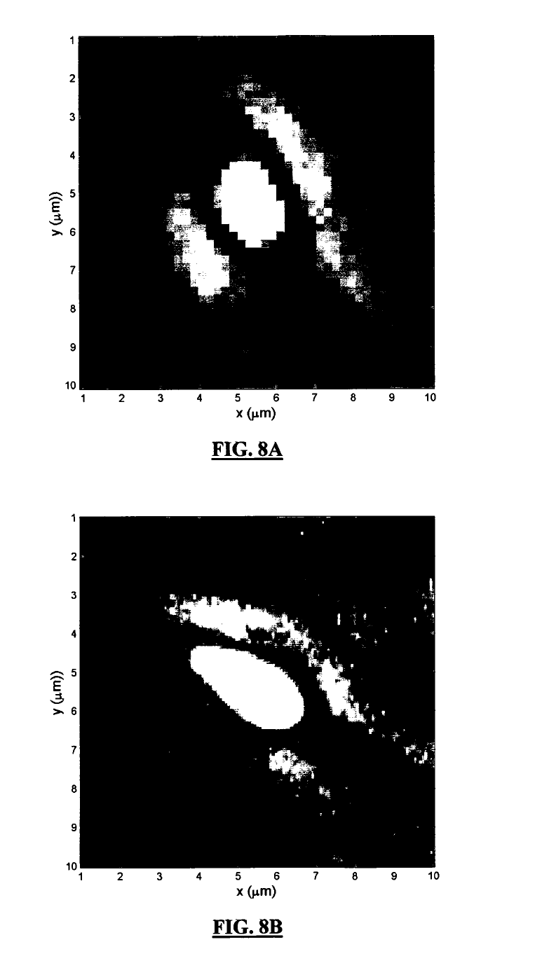

FIG. 8 shows a comparison of high resolution imaging of live sperm cells. FIG. 8B is taken at a higher spatial resolution than FIG. 8A for the same magnification.

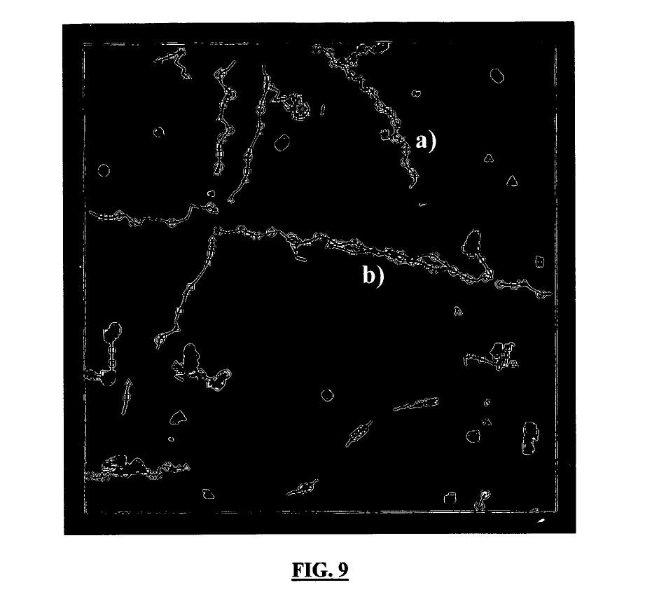

FIG. 9 shows a sample image taken from an apparatus according to an embodiment of the invention, illustrating the identification and tracking marking applied to the image.

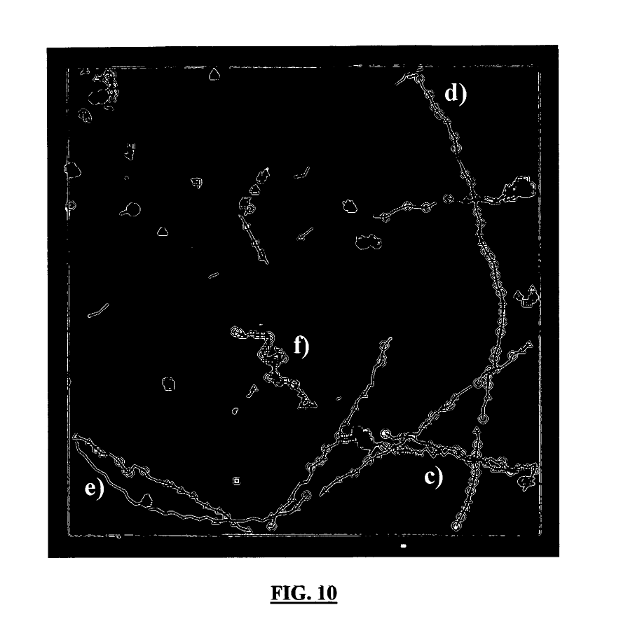

FIG. 10 shows another sample image taken from an apparatus according to an embodiment of the invention, illustrating the identification and tracking marking applied to the image.

The present inventors assembled a CASA based upon existing microscopes and digital video cameras used in particle image velocimetry (PIV), a technique used in aerodynamics and fluid mechanics (Green et al 2000). Digital video images of live semen samples were recorded using the CASA apparatus. These samples were from a combination of fresh and frozen, donor and patient samples. Off-line analysis of the digital images was performed, and sperm cell motility and morphology data were successfully obtained from the images. Using the embodiment of the invention it was possible therefore to provide simultaneous morphology and motility analysis of a live semen sample.

An apparatus 10 according to an embodiment of the invention is shown in FIG. 1.

Optical microscope 12 is provided with phase contrast optics 14. A specimen chamber 16 is mounted within a temperature controlled enclosure 20 on a motorized stage 18 on base 19. For human sperm, a 20 micrometer chamber depth is required to conform with WHO guidelines.

The apparatus has an image recording system for recording a sequence of images from the microscope. In this embodiment, a monochrome, digital video camera 22 is attached to the microscope. Camera 22 is connected to a dedicated frame grabber 24. A computer 26, with a display and/or printing interface is connected to the microscope stage 18. The computer is for image capture and analysis (including control of the microscope) and for running the software for the motility and morphology algorithms.

A feature of the system is that the optical and camera system is able to record images of adequate spatial resolution for a sufficiently accurate morphological analysis. Furthermore, the camera has a high enough frame rate so that the kinematics of the moving objects may be resolved. This is explained in more detail below.

The preferred minimum spatial resolution required by the camera and microscope is that to satisfy the Nyquist sampling theorem. For example, to resolve the morphological features of a human sperm cell a typical microscope with a ×20 objective with a numerical aperture of 0.4 requires a camera with pixel size of 7.4 micrometers square with a sensor resolution of 1000×1000 pixels. Careful matching of the microscope and camera in this way optimises the accuracy of the morphological measurements, and a less well matched set-up will compromise the system performance. For free moving cells in a fluid (such as live sperm in a semen sample) the depth of field of the microscope and camera system have to be as large as possible without compromising the spatial resolution. For the frame rate of the camera the Nyquist sampling theorem is also appropriate to use as a guideline, and for human sperm a camera frame rate of 30 Hertz is preferred to sample the spermatozoa kinematics correctly.

The microscopes employed in the study were standard laboratory upright microscopes (e.g. Olympus BH12) fitted with phase contrast rings and ×20 and ×40 objectives. The microscopes were fitted with C-mount adapters for attaching the video camera 22. One camera used was a Kodak Megaplus ES1.0, 8-bit monochrome digital video camera, which is a full frame camera with a 1k×1k pixel CCD array and maximum framing rate of 30 fps. For image recording a high-performance desktop PC running Windows 98 was used, and this was fitted with a National Instruments PCI-1424 digital video capture card and National Instruments PC-TIO-10 counter-timer card to control the camera frame rate. The system was programmed using LabVIEW, which provided a simple graphical user interface, and allowed the correct exposure and recording parameters to be set. The camera gain and shutter speed and the microscope illumination level were set to avoid saturation of the images while providing as sharp an image of a sperm cell as possible. In this embodiment, up to 72 images could be captured for each digital video sample, and the LabVIEW system saved the digital images in uncompressed TIFF format. Additional images were taken using a Nikon D1X high resolution, digital colour stills camera. This camera has a CCD array of 3k×2k pixel resolution with 8-bit depth on each RGB channel.

Digital video recordings were made of semen samples provided for analysis by 11 men undergoing infertility investigations in addition to 8 fresh or frozen samples provided by research donors attending the donor insemination programme. These samples were each analysed for sperm concentration, motility and morphology using the standard manual (WHO, 1999) techniques. In addition, the samples were then observed using the above-described CASA apparatus. A total of 6 to 8 full fields of view of 2.4 seconds duration at 30 fps and shutter speed 5 ms were captured for later analysis. Images were taken for ×20 and ×40 magnification. Spermatozoa were observed in a 20 μm depth Microcell chambers to allow the spermatozoa the ability to display their full range of motion characteristics. For some of the semen samples digital still images were taken using the Nikon camera set to a sensitivity of ISO800 and a shutter speed of 1/100 s.

As shown in FIG. 2, the overview of the process is image capture 202, followed by image analysis 204 and then data analysis 206, followed by data presentation 208.

With reference to FIG. 3, for each test, a semen sample is initially placed in a chamber and the chamber placed in the microscope. The camera is focussed at step 302, using the microscope optics, on any user defined focal plane within the chamber, and microscope and illumination and camera exposure levels are set at step 304. Image capture may then take place. A suitable image capture rate is then set (for example 30 Hz as discussed above) at step 306 and the duration of image capture is set to comply with WHO guidelines (a minimum duration of least 0.8 seconds is required by those guidelines). After one field is captured (step 308) the computer moves the microscope stage (step 310) to a new field and the process is repeated, typically for six fields. The image data for each frame for each field is stored on the computer.

The computer system then executes the image analysis methodology which comprises morphology and motility analysis processes. The software for carrying out the image analysis was programmed on a PC using MATLAB. An overview of the process is shown in FIG. 4, showing an object identification stage 402 and an object tracking stage 404.

FIG. 5 illustrates in more detail the steps taken during the object identification stage 402.

In order to simultaneously measure motility and morphology of a spermatozoon in a confident manner the image data should first be split into object (essential) data and background (non-essential) data. Every spermatozoon that exists within the image depth of field should be found, measured and then followed through an image sequence. To the human eye the most distinctive characteristic of a spermatozoon is the head and it was therefore appropriate to use this feature for automated spermatozoon detection. There are very distinguishing morphological aspects of a head that allow it to be consistently filtered from similar sized and orientated structures in the sample (e.g. germ cells or leucocytes). In particular the area between the acrosome and the midpiece is distinctive both in contrast and in shape and it is well isolated by the head membrane, preventing illumination profile contamination even when other cells are contiguous to the spermatozoon perimeter. Additional or alternative recognisable features of a sperm cell are: a nominal area; a nominal length to width ratio; nominal intensity profile along its major axis. These attributes make for a confident, repeatable morphological characteristic for a motile spermatozoon. It is then a straightforward step to build a morphological filtering routine to extract these structures from the image plane. Such a routine will be arrived at in a straightforward manner by a person skilled in the art.

The first frame in the recorded sequence of a given field is selected. FIG. 5 shows the steps taken. Possible object data of interest on this frame are identified by their change of gradient on the grey level digital image (step 502). Perimeters of each object are located, and clumped objects have their perimeters located using a skeletal algorithm so that each potential cell structure is isolated (step 504). Cells of interest are then filtered out from this set of object data (step 506) as follows. A weighted sum of the characteristic features for each object in the sample is then used to identify sperm cells in the image. In step 508, the filtered data are then re-analysed on a cell-by-cell basis to extract detailed cell morphology data (for example length, width, area, shape). The next frame of the same sequence is then selected (step 510), and the above analysis is repeated until the last frame of the recorded sequence has been analysed.

An important factor in this embodiment is the application of image processing algorithms in an order that exposes the object characteristics of interest. The skilled person will realise that image processing (and especially morphological assessment algorithms) can yield the same result using many different techniques. The techniques described in broad terms here may therefore be replaced by other, equivalent techniques.

Sequential identification of the individual structures in the sample for each frame is performed and a decision is made as to whether it is important. This can be done because when imaged correctly spermatozoa have very particular characteristics (illumination profile, shape and size) that are not specifically shared with any other extra-cellular material extant in the sample.

The illumination profile of an object is the intensity profile on the image plane that describes the spermatozoon. The intensity distribution of the nucleus part of the sperm head is very distinctive and a simple morphological filter used to identify this artefact. The artefact has a centre point of maximum intensity with a nearly axis-symmetric domical intensity profile, similar to a bell curve. The acrosome shape and acrosome intensity distribution are also repeatable and distinguishable and can be used to identify the sperm head, particularly when a high spatial resolution image is obtained.

The inventors consider that the illumination footprint is the best initial filter for identifying the sperm since this places no restrictions on the physical shape or physical size of the objects to be measured. It also works when other cells are contiguous with the head perimeter. This is a more rigorous procedure since it means that all spermatozoa are measured irrespective of distortion or irregularities, yielding a better statistical measurement. Previous CASA systems are only capable of identifying sperm that are of a particular size and shape, and these limits must be controlled very closely for successful operation.

Typically, cameras will always generate dark current (or noise) and so it is to be expected that not all illumination profiles identified using the initial filter described above are actually the heads of spermatozoa. Therefore a shape filter is used to extract the perimeter of the objects located above to see, for each object, if it is contained in one body (that is the perimeter of the structure contains an acrosome and a nucleus part). The shape should also take on the generic features of a sperm head (a pointed head with orbicular body), obtained once again through morphological filtering.

The final filter routine performed measures the size of the object data. This is much less important than the other characteristics but is used in the weighted voting procedure to decide whether the object identified is a sperm cell or not. For human sperm the length and width of the head, midpiece (between head and tail) and principle piece (tail) are well known from previous research. The present system can then measure these features and judge their similarity. The advantage is that this final filter can be much more relaxed than the size filtering imposed by other CASA systems.

On the basis of the results from each filter, it is possible to make a confident appraisal of each individual object using a weighted voting procedure. In the event that the voting procedure does not provide a conclusive decision for a particular object, then the object is checked using an algorithm for identifying tail protrusions and/or object movement.

The objects that are not categorized as spermatozoa may still be important and some classification as to their nature is pursued. Therefore the system measures each remaining structure and logs its pertinent characteristics. Immature germ cells are automatically identified by the system (since these are the most frequent of residual structures) and leukocytes are identified also.

Usually the detailed morphological information for a cell is taken from the best in-focus picture of the cell. Once the detailed morphological analysis has been performed for each cell of interest in the frame sequence of image data, the motility analysis may then proceed using the morphological data obtained. FIG. 6 shows the steps taken during the motility analysis.

The first image pair (the first and second frames of image data) in the sequence is used to make an initial projection of each cell track based upon known cell morphology and orientation and a simple tracking scheme, for example a nearest neighbour method. Then any existing cell tracks are extended into the next frame using the available morphology and cell track information. Any new tracks appearing in any two successive frames are started off and continued appropriately. This is completed for all frames, and morphological and kinematic data are used to close any broken tracks where the cell might have moved out of focus between frames. The frames are then re-examined to identify non-motile cells and other non-motile objects, which are then classified accordingly. Each individual cell track is then examined and all useful morphological data along the track are used to build up the most complete morphological characterization of that cell. Finally, referring back to FIG. 2 (steps 206 and 208), the system analyses the cell track and morphological data and provides a representative set of sample based statistics for the analysis regarding cell morphology and motility, for example the number or proportion of morphologically normal, motile cells in the analysis.

Looking at the motility analysis in more detail, an algorithm was constructed that would follow each spermatozoon through the object data sequence. A known straightforward tracking methodology is to track the nearest neighbour between fields/frames. A nearest neighbour analysis is what existing CASAs use. However such a nearest neighbour scheme is not appropriate due to sperm collisions, which would lead to broken tracks and other anomalies that skew the statistics unless accounted for. Instead, nearest neighbour analysis provides the basis for the present methodology.

The methodology employed for motility measurement was to combine the nearest neighbour analysis with the high quality morphological data obtained in step 402. The tracking technique matches kinematic properties of a moving spermatozoon (i.e. its velocity and trajectory) and also its morphological qualities, already measured and stored when establishing object data. There are significant benefits to this approach when the sample is highly populated and collisions are commonplace. By using the morphological properties of the head it was possible to distinguish between a number of spermatozoa even in close proximity, thus making it a successful tracking algorithm even when collisions and crossings were prevalent. Since the head shapes were measured in each frame, the morphological algorithm could then seek out the best profile to measure the shape characteristics with respect to focus and attitude of the sperm cell.

Looking at FIG. 6, this drawing shows a flow diagram layout of the object tracking algorithm for step 404. A frame is selected at step 602 and routine 604 is carried out for that image data. For a cell of interest in the frame, the morphological data for that cell is uploaded (step 606). The purpose of the tracking algorithm may be thought of simply as joining the dots between frames. The movement of the sperm cell is predicted in step 608. Based on the detailed morphological data captured from one frame, the orientation and shape of the sperm head in that frame is known. Thus it is possible to reassess the prediction of step 608 to more accurately project where the spermatozoa is going to travel between frames (step 610), while it is already known what it actually looks like (head perimeter-shape, head area, head length, head width, acrosome area, DNA area etc.). Finally, for that cell in that frame, a track decision is made (step 612) to assess the motion of the cell from the previous frame. Note that as the track gets longer the kinematics of the spermatozoon are better estimated, making further tracking easier. Steps 606-612 are repeated (step 614) in the frame. Next, the routine is repeated for each frame of the sequence (616).

From an optical perspective the depth of field of the microscope causes some spermatozoa to come in and out of focus. In preferred embodiments there are in-built procedures to check that if a spermatozoa does ‘disappear’ then the residual structures are checked (where the data would reside) and the track is re-connected. Throughout a complete track all the required kinematic data can be extracted and the morphological data can be measured also.

There are known techniques that one skilled in the art of image analysis processing will be able to use to provide specific algorithms for suitable image analysis. For example, thresholding with filters may be used to seek out specific spatial and illumination shape profiles.

The above-described image analysis methodology was applied to the digital image recordings of donor and patient semen samples collected at the Andrology Laboratory in Sheffield, UK. In this example, the Kodak ES1.0 camera was used. Its spatial resolution was adequate to just distinguish between the individual profiles of spermatozoa heads, thus allowing the tracking algorithm to operate. It was seen that the measurement errors caused by camera digitisation affect the nominal head width and head length by ±4.8% and ±7.5% error respectively.

FIG. 7 shows normalized frequency distributions of length/width ratio for progressively motile sperm having an average speed of greater than 5 micrometers per second for samples from a range of donors and patients. From this graph there are clear variations of the frequency distributions of the head length/width ratio of the individual samples, and the camera measurement error is not sufficiently large to make this difference irrelevant. The data was consistent with WHO (1999), although there is variation between individuals giving further merit to the proposition that the ability to make simultaneous motility and morphology measurements will be clinically useful. A note of interest is that the semen sample from Patient 1 (FIG. 7) contained a large number of immature germ cells, which appear morphologically as distinctive circles and morphological filters were written to detect these. Since the presence of germ cells in an ejaculate is a marker of testicular dysfunction, the ability to detect these at the same time that motility and morphology is being measured is a distinct advantage.

FIG. 8 shows a comparison of high resolution imaging of live sperm cells. FIG. 8B (taken using Nikon D1X camera) has at a higher spatial resolution than FIG. 8A (taken using Kodak Megaplus ES1.0 camera) but at the same magnification. The pixellation in FIG. 8A is much clearer than in FIG. 8B, the definition of the shape of the head of the sperm in FIG. 8B being much clearer in FIG. 8B than in FIG. 8A.

FIGS. 9 and 10 show overlaid sample output images from the software and digital camera.

FIG. 9 illustrates the identification and tracking marking applied to the image. Only the tracks of motile sperm are shown (for clarity of presentation) and these are displayed as solid lines. Note that the output from the apparatus uses false colour to identify tracks of cells with different morphology scores, but that cannot be reproduced here. Points along each track where the sperm cell has presented a good enough image to obtain morphology data are represented by circles, with the quality of that morphology data represented by the false colours mentioned above. It may be seen that, along any particular track, each individual sperm cell sometimes presents a good profile for morphological measurements, and at other times (say as a result of swimming out of focus, or of poor orientation relative to the camera) presents a less good opportunity for gathering morphological data. An example of this is shown for track a) of FIG. 9.

By collecting morphology data at several points along a track, a more complete morphological picture of each individual cell can be obtained. FIG. 9 also shows a good example of how the algorithm uses morphological data to differentiate between the tracks of two (or more) individual sperm cells which cross paths, move close to one another or collide. This can be seen at point b) in the FIG. 9, where two sperm tracks run almost alongside each other, but also cross regularly. Because each cell has individual morphology, then the algorithm can separate the two tracks, as a direct consequence of performing morphological measurements and motility measurements on the same live sample. This process was not previously possible using existing CASA machines.

In FIG. 10, points c) and d) also show the algorithm differentiating between tracks that cross. Also on this figure is an example of a long sperm track (track labelled d)) where a great deal of morphology data is available, and another track (track f)) where the sperm has moved much less distance. This shows that the present system can supply quantitative data on motility levels (and corresponding morphologies) of individual cells. Point e) in FIG. 10 is also of interest, in that it shows a successfully tracked sperm cell which has been slightly out of focus, and therefore has presented relatively poor morphology data, except for a few points along the track. The important feature here is that the tracking algorithm continues to operate even when morphology data is poor.

The above embodiments have been described by way of example only. Modifications of these embodiments, further embodiments and modifications thereof will be apparent to the skilled person on reading this disclosure and are within the scope of the invention.

### LIST OF REFERENCES

The following non-patent publications are referred to above. The content of each of these publications is hereby incorporated by reference in its entirety.
Aitken, R. J., Sutton, M., Warner, P., and Richardson, D. W., (1985) Relationship between the movement characteristics of human spermatozoa and their ability to penetrate cervical mucus and zona-free hamster oocytes. J. Reprod. Fertil., 73: 441-449.
Clements S., Cooke I. D. and Barratt, C. L. R. (1995) Implementing comprehensive quality control in the andrology laboratory. Hum. Reprod., 10, 2096-2106.
Ellington, J. E., Evenson, D. P., Wright, R. W., Jones, A. E., Schneider, C. S., Hiss, G. A., & Brisbois, R. S., (1999) Higher quality human sperm in a sample selectively attach to oviduct (Fallopian Tube) epithelial cells in vitro. Fertility and Sterility. 71: 924-929.
Green, R. B., Doolan, C. J. & Cannon, R. M. (2000) Measurements of the orthogonal blade-vortex interaction using a particle image velocimetry technique. Experiments in Fluids, 29: 369-379
Katz, D. F., Morales, P., Samuels, S. J., Overstreet, J. W., (1990) Mechanisms of filtration of morphologically abnormal human sperm by cervical mucus. Fertility and Sterility 54: 513-516
Liu, D. Y and Baker, H. W. G., (1992) Morphology of spermatozoa bound to the zona pellucida of human oocytes that failed to fertilise in vitro. J. Reprod. Fert., 94: 71-84
Martin, L. C., (1966) The theory of the microscope, pub. Blackie & Son, London.
Matson, P. L., (1995) External quality control assessment for semen analysis and sperm antibody detection: results of a pilot scheme. Hum. Reprod., 10, 620-625.
Mortimer, D., Pandya, I. J., and Sawers, R. S., (1986) Relationship between human sperm motility characteristics and sperm penetration into human cervical mucus in vitro, J. Reprod. Fertil, 78: 93-102
Mortimer, D. (1994) Practical Laboratory Andrology, Oxford University Press, NY, 393
Swan, S. E., and Elkin, E. P., (1999) Declining semen quality: can the past inform the present? BioEssays. 21: 614-621.
World Health Organisation (1999) WHO laboratory manual for the examination of human semen and sperm-cervical mucus interaction, 4^{th} edn, Cambridge University Press, Cambridge, UK.

1-38. (canceled)

39. A method for determining the morphology and motility of a population of cells in vitro including the steps:
(a)(i) capturing a first frame of image data of said population and identifying a part or parts of the image data corresponding to a cell or cells of interest;
(a)(ii) capturing a second frame of image data of said population and identifying a part or parts of the image data corresponding to a cell or cells of interest;
(b)(i) determining the characteristic morphology of the cell or cells of interest from the first and/or second frame;
(c)(i) tracking the cell or cells of interest by identifying each cell of interest by its characteristic morphology and determining the relative displacement, in the second frame compared to the first frame, of said cell or cells of interest.

40. A method according to claim 39 wherein the first and second frames are adjacent frames in a series of more than two frames of image data captured of said population, the method further including, for each frame of said series, the steps:
(a)(iii) identifying a part or parts of said image data corresponding to the cell or cells of interest;
(b) (ii) determining the characteristic morphology of said cell or cells identified in step (a)(iii);
(c)(ii) tracking the cell or cells of interest by identifying each cell of interest by its characteristic morphology and determining the relative displacement, in said frame compared to the previous frame in said series, of said cell or cells of interest.

41. A method according to claim 39 including the step of determining a value for the motility of the cell or cells of interest, based on the relative displacement of the cell or cells of interest.

42. A method according to claim 39 including the step of determining the amount or relative amount of the population of cells having a motility at or above a threshold motility value.

43. A method according to claim 42 including the step of classifying the cell or cells of step (b)(i) or step (b)(ii) as morphologically normal or morphologically abnormal.

44. A method according to claim 43 including the step of determining the amount or relative amount of the population of cells being morphologically normal.

45. A method according to claim 43 including the step of determining the amount or relative amount of the population of cells:
being morphologically normal, and
having a motility at or above a threshold motility value.

46. A method according to claim 39 for carrying out a first determination of morphology and motility on a first area of a sample of cells, the method including the step of carrying out a second and, optionally, further, determinations of morphology and motility on a second and, optionally, further, areas of the sample.

47. A method of processing image data captured from a population of cells in vitro in order to determine the morphology and motility of the cells, the image data including a first frame of image data of said population and a second frame of image data of said population, the method including the steps:
(i) determining the characteristic morphology of the cell or cells of interest from the first and/or second frame; and
(ii) tracking the cell or cells of interest by identifying each cell of interest by its characteristic morphology and determining the relative displacement, in the second frame compared to the first frame, of said cell or cells of interest.

48. A method according to claim 47 wherein the first frame of image data is processed to identify illumination intensity distributions of interest having one of a plurality of characteristic profiles.

49. A method according to claim 48 wherein one of the characteristic profiles is a first characteristic profile having a centre point of a relatively high intensity surrounded by a substantially symmetrical gradual reduction in intensity.

50. A method according to claim 48 wherein the parts of the image data corresponding to the illumination intensity distributions of interest are further processed to identify cell perimeter features surrounding one or more of said illumination intensity distributions of interest.

51. A method according to claim 48 wherein an object of interest is identified, the method further including the step of determining one or more dimensions or relative dimensions of an object.

52. A method according to claim 51 wherein said dimensions or relative dimensions are compared to one or more predetermined ranges of corresponding dimensions or relative dimensions.

53. A method according to claim 39 further including the step of determining whether said object is a cell to be tracked or not and:
if said object is a cell to be tracked, assigning a tracking identity to it; or
if said object is not a cell to be tracked, assigning a residual object identity to it.

54. A method according to claim 39 further including the step of determining a characteristic morphological value for said cell to be tracked.

55. A method according to claim 39 repeated in order to identify all cells to be tracked and all objects not to be tracked in a frame of image data.

56. A method according to claim 55 repeated for the second and/or subsequent frames.

57. A method according to claim 39 wherein when the tracks of two cells of interest intersect, the cells and their tracks are identified before and after the intersection by their characteristic morphologies.

58. A method according to claim 39 wherein when a cell of interest is identified in one frame and not identified in the next frame, the cell being identified in a subsequent frame, the method further includes the steps of calculating tracking data to connect the track of the cell through the frames.

59. A method according to claim 39 further including the step of determining a motility characteristic for a tracked cell.

60. A method according to claim 59 further including determining an overall figure of merit for the sample indicative of the number or proportion of morphologically normal cells with normal motility.

61. A method according to claim 39 wherein image capture is performed using digital imaging means providing a frame resolution or an effective frame resolution of at least 0.5×10⁶ pixels.

62. A method according to claim 61 wherein the rate of image capture for a series of frames is at least 20 Hz.

63. A method according to claim 39 wherein the cell or cells of interest are spermatozoa.

64. A method according to claim 63 wherein the cell or cells of interest are human spermatozoa.

65. A method according to claim 39 further including the step of diagnosis based on the determination of the morphology and motility of the population of cells in vitro.

66. A method according to claim 65 wherein the step of diagnosis is based on a value of the amount or relative amount of cells categorised as morphologically normal and having a motility at or above a threshold motility value.

67. Apparatus for determining the morphology and motility of a population of cells in vitro, the apparatus including:
imaging means for capturing first and second frames of image data of said population and identifying a part or parts of the image data corresponding to a cell or cells of interest;
computation means for determining the characteristic morphology of the cell or cells of interest from the first and/or second frame, tracking the cell or cells of interest by identifying each cell of interest by its characteristic morphology and determining the relative displacement, in the second frame compared to the first frame, of said cell or cells of interest.

68. Apparatus according to claim 67 wherein the imaging means is digital imaging means providing a frame resolution or an effective frame resolution of at least 0.5×10⁶ pixels.

69. Apparatus according to claim 68 wherein the imaging means is arranged to capture a series of frames at a rate of at least 20 Hz.

70. Apparatus according to claim 67 wherein the imaging means includes phase contrast optics.

71. A computer system operatively configured to carry out the method according to claim 39.

72. Computer programming code for operatively configuring a computer system to carry out the method according to claim 39.

73. A data carrier having recorded on it computer programming code according to claim 72.
